(19) United States
(12) Patent Application Publication
(10) Pub. No.: US 2025/0241535 A1
(43) Pub. Date: Jul. 31, 2025

(54) METHOD AND SYSTEM FOR DETERMINING HEARTBEAT CHARACTERISTICS

(71) Applicants: Andrey Viktorovich FILIMONOV, Kamenki (RU); Harman Becker Automotive Systems GmbH, Karlsbad-Ittersbach (DE)

(72) Inventors: Andrey Viktorovich FILIMONOV, Kamenki (RU); Ivan Sergeevich SHISHALOV, Nizhniy Novgorod (RU); Andrey Sergeevich SHILOV, Nizhny Novgorod (RU); Roman Aleksandrovich ERSHOV, Nizhny Novgorod (RU)

(21) Appl. No.: 18/854,493

(22) PCT Filed: Apr. 5, 2022
(86) PCT No.: PCT/RU2022/000104

§ 371 Date: Oct. 4, 2024

(51) Int. Cl. A61B 5/00; G06T 7/00

(52) U.S. Cl. CPC A61B 5/0082; A61B 5/0064; A61B 5/0075; A61B 5/0077; G06T 7/0012; G06T 2207/10004; G06T 2207/10024; G06T 2207/30088; G06T 2207/30104

(57) ABSTRACT

The disclosure relates to a method including: capturing a first set of image frames, wherein the first set of image frames includes a representation of a user's face; identifying at least one skin patch of the user's face that is represented in the first set of frames; determining a light source configuration and transmitting the light source configuration to a first light source; illuminating, by the first light source, the at least one skin patch according to the light source configuration; capturing a second set of image frames, wherein the second set of image frames includes a representation of the at least one skin patch illuminated by the first light source according to the light source configuration; and processing one or more of the second set of image frames using remote photo-plethysmography, rPPG. The disclosure also relates to a system configured to perform the method.

### CROSS-REFERENCE TO RELATED APPLICATIONS

The present application is a U.S. National Phase of International Patent Application Serial No. PCT/RU2022/000104 entitled “METHOD AND SYSTEM FOR DETERMINING HEARTBEAT CHARACTERISTICS,” filed on Apr. 5, 2022. The entire contents of the above-referenced application(s) are hereby incorporated by reference for all purposes.

### FIELD

The present disclosure relates to a method and a system for determining heartbeat characteristics, such as heartbeat rate.

### BACKGROUND

Heartbeat characteristics are a source of human physiological and psychological information. Parameters like heartbeat rate, heartbeat rate variability and heart inter-beat intervals can be indicative of a person's health and wellbeing. Such parameters can be measured using sensors that are in contact with a user's skin. One example is electrocardiogram which a heart's electrical activity is measured using electrodes placed on the skin of the user.

Another method of measuring heartbeat characteristics is photoplethysmography (PPG). PPG is an optical method to detect volumetric changes in blood based on measurements at the surface of the skin. Common PPG methods usually require some form of contact with the human skin (e.g., ear, finger).

A particular form of PPG is remote PPG (rPPG). RPPG is a noncontact video-based method that monitors the change in blood volume by capturing pixel intensity changes in images of the skin. RPPG can be used in environments where users are stationary, at least temporarily, and can be monitored by a camera. One such environment is the interior of a vehicle. Measuring heartbeat characteristics of a driver of a vehicle allows detecting conditions that may be detrimental to the driver's ability to drive, for example stress and tiredness.

In the interior of a vehicle, however, the accuracy of rPPG is an issue. This may be caused by changing light conditions. For example, the vehicle may be used at day or at night, and at varying conditions (sunshine, rain, in tunnels etc.). Also, there are light sources within the vehicle that may or may not be active. Thus, portions of the skin of a driver that could be used for rPPG may be exposed to unpredictable and ever-changing light conditions.

The present disclosure aims to address these and other issues.

### SUMMARY

The present disclosure provides a method and a system for determining heartbeat characteristics, such as heart rate, using rPPG. The method and system aim to provide improved accuracy of rPPG by identifying and illuminating a specific area in the face of a user while performing rPPG on that area. The method and system are particularly useful in an environment where light conditions are unpredictable and variable, such as in a vehicle.

To achieve these and other desired effects, according to one aspect of the present disclosure, there is provided a method including: capturing a first set of image frames, wherein the first set of image frames includes a representation of a user's face; identifying at least one skin patch of the user's face that is represented in the first set of frames; determining a light source configuration and transmitting the light source configuration to a first light source; illuminating, by the first light source, the at least one skin patch according to the light source configuration; capturing a second set of image frames, wherein the second set of image frames includes a representation of the at least one skin patch illuminated by the first light source according to the light source configuration; and processing one or more of the second set of image frames using remote photo-plethysmography, rPPG.

Initially, the method may identify a skin patch in the user's face. The term ‘skin patch’ is used herein to designate an area in the user's face that may be suitable for rPPG. Such areas may be located in regions of the user's face where the skin is relatively even, for example in regions of the forehead or cheeks. The location of the skin patch can be identified based on its known relative location to facial features, such as the user's eyes. Such facial features can be identified using known feature detection methods. Accordingly, by first detecting pixels representing facial features in the first set of image frames, it is possible to identify pixels representing skin patches suitable for rPPG, as will be explained in more detail below.

In subsequent steps, the method may determine a light source configuration, and illuminating the identified skin patch using a light source configured according to the determined light source configuration. The light source configuration may be determined so as to focus light emitted from the light source onto the skin patch. Also, the light source configuration may be determined so as to avoid illuminating areas of the user's face that are too close to the user's eyes, in order to avoid any distraction or irritation.

The light source configuration may relate to different configurable parameters of the light source, including, but not limited to the angle, direction, diameter, light intensity, wavelength and duration of emitted light beams. The light source configuration may also take into account existing environmental light (e.g., daylight, background illumination in a vehicle). Further, the light source configuration may be dynamically adapted to changes in the environmental light.

The parameters may be set so as to optimise the results of rPPG based on light reflected from the skin patch. To this end, the method may include capturing a second set of image frames that include a representation of the illuminated skin patch. Accordingly, the method according to the present disclosure can provide improved rPPG results by enabling the execution of rPPG based on light reflected from selected, illuminated regions of the user's face.

In one of a number of embodiments, the capturing of the first and second sets of image frames is performed by a single camera. Accordingly, the method may be implemented with relatively few components, making it particularly suitable for environments where space is limited, such as the interior of a vehicle or car.

The camera may include a red-green-blue (RGB) sensor, wherein the capturing of the first and second sets of image frames includes capturing images in the visible light spectrum using the RGB sensor. In an alternative embodiment, the camera may include a near-infrared, NIR, sensor, and an RGB sensor, wherein the capturing of the first set of image frames includes capturing images in the NIR spectrum using the NIR sensor, and wherein the capturing of the second set of image frames includes capturing images in the visible light spectrum using the RGB sensor. Using an additional NIR sensor may improve the accuracy of identifying suitable skin patches for rPPG.

In another embodiment, the capturing of the first set of image frames is performed by a first camera, and the capturing of the second set of image frames is performed by a second camera. Using two separate cameras may improve the accuracy of skin patch detection and rPPG. For example, the first camera may be arranged at a location that is particularly suitable for skin patch identification, whereas the second camera may be arranged at a location that is particularly suitable for rPPG. Alternatively, the first and second cameras may be arranged adjacent to one another and included in a single housing to obtain compact dimensions.

The first camera may include an NIR sensor to capture the first set of image frames in the NIR spectrum. The second camera may include an RGB sensor to capture the second set of image frames in the visible light spectrum. The combination of a camera with an NIR sensor and a camera with an RGB sensor provides highly accurate rPPG results.

While using an NIR sensor in itself may already improve the identification of suitable skin patches for rPPG, the method may further include illuminating the user's face with a NIR light source. Using an NIR light source to illuminate the user's face and identifying the skin patch in the NIR spectrum may reduce or even eliminate any interference from environmental light. Also, NIR light has the advantage of being invisible to the naked eye. Further, NIR light is unlikely to interfere with other light-sensitive systems that may be present, for example in a vehicle.

Whilst some of the described embodiments include an NIR sensor to capture images in the NIR spectrum, or a NIR light source, the present disclosure is not limited in this respect. Other wavelengths, in particular other infrared wavelengths, and corresponding sensors and light sources, may be used.

In one embodiment, identifying the at least one skin patch includes detecting one or more facial features represented in the first set of image frames, and identifying the at least one skin patch based on a location of the detected one ore more facial features. Detecting the presence and location of facial features may facilitate the identification of skin patches. For example, if regions of the forehead are considered particularly suitable for rPPG, such regions may be identified by their relative location to the eyes. The detection of facial features in the first set of images frames may be implemented using known features detection methods.

Also, the method may include identifying two or more skin patches, and selecting one or more of them for rPPG. In other words, ‘candidate’ skin patches for rPPG may be identified, and those most suitable for rPPG may be selected. The selection may be based on parameters such as the location or size of the candidate skin patches, or the signal-to-noise ratio, which may be derived from the image frames in which the skin patches are represented.

Further, the method may include selecting at least some of the second set of image frames for rPPG processing. The selection may be based on one or more quality criteria, for example a signal-to-noise ratio. By performing a quality-based selection of image frames before performing rPPG, the results of rPPG processing may be improved even more.

The method may further include determining one or more heartbeat parameters based on the results of the rPPG processing and generating a control signal based on the one or more heartbeat parameters to initiate an action associated with the one or more heartbeat parameters. Accordingly, the method enables determining different physiological or psychological states of the user based on the heartbeat parameters, and taking appropriate actions. For example, the user may be a driver of a car, and the heartbeat parameters may indicate a state of fatigue. Thus, the method may be used to generate a warning signal to the driver, inviting him to take a break, or even to actively intervene, for example by reducing the speed of the car.

According to another aspect of the present disclosure, there is provided a system configured to perform any of the steps of the method according to the present disclosure. In particular, there is provided a system including: at least one camera for capturing a first set of image frames, wherein the first set of image frames includes a representation of a user's face; a facial feature detector for identifying at least one skin patch of the user's face that is represented in the first set of image frames; a light source configurator for determining a light source configuration; and a first light source for illuminating the at least one skin patch according to the light source configuration; wherein the at least one camera is configured to capture a second set of image frames, wherein the second set of image frames includes a representation of the at least one skin patch illuminated by the first light source according to the light source configuration, and wherein the system further includes a remote photo-plethysmography, rPPG, system for processing one or more of the second set of image frames using rPPG.

In one embodiment, the at least one camera may include an RGB sensor for capturing the first and second sets of image frames. Alternatively, the at least one camera includes an NIR sensor for capturing the first set of image frames in the NIR spectrum, and an RGB sensor for capturing the second set of image frames in the visible light spectrum.

In another embodiment, the at least one camera includes a first camera for capturing the first set of image frames, and a second camera for capturing the second set of image frames. The first camera may include an NIR sensor for capturing the first set of image frames in the NIR spectrum, and the second camera may include an RGB sensor for capturing the second set of image frames in the visible light spectrum.

The first light source may include one or more light emitting diodes (LEDs). LEDs consume relatively little energy and space and are therefore well suited for integration in an in-vehicle system. Alternatively, the first light source may include a liquid crystal display (LCD) projector, a digital light processing (DLP) projector or a charged-coupled device (CCD) projector. Light from such projectors can be precisely focused onto the identified skin patch.

The first light source may be configurable by electronic, electromechanical and/or mechanical means. The first light source may be configurable to adjust its field of projection so as to include the user's face and, in particular, the at least one skin patch.

In yet another embodiment, the first light source may include a plurality of light emission sources at different locations, thereby to improve the illumination of the skin patch. The presence of a plurality of light emission sources at different locations also provides redundancy and enables maintaining an illumination of the skin patch if one of the light emission sources fails or is temporarily obstructed.

In some embodiments that include a camera with an NIR sensor, the system may include an additional light source for illuminating the user's face with NIR light.

In an embodiment, the first light source is controllable to illuminate two or more skin patches on the user's face separately and/or independently of one another. To this end, the first light source may include a plurality of light emitting sources. The light emitting sources may be arranged adjacent to one another, in a single device, or at different locations. Accordingly, it is possible to identify two or more skin patches, and to focus separate light beams onto the skin patches. The light emitting sources may be controlled to illuminate the skin patches at the same time, consecutively, or alternatingly. By capturing light reflected from the skin patches thus illuminated, more data for rPPG processing can be obtained, thereby further improving rPPG accuracy.

Also, identifying and illuminating two or more skin patch analysis reduces the vulnerability to external conditions which may obstruct a given skin patch, for example due to movements of the user, clothes (e.g., hats) or sudden changes in environmental lighting (e.g., shadows).

Some or all of the plurality of light emission sources may be individually configurable. For example, some of the plurality of light emission sources may be controlled to illuminate one skin patch with a first intensity or wavelength, while others of the plurality of light emission sources may be controlled to illuminate another skin patch with a second (different) intensity or wavelength. This enables individually compensating for different light conditions at each skin patch, resulting in higher quality images of the skin patches and improved rPPG accuracy.

The at least one camera and the first light source may be arranged at a distance from one another, for example at suitable locations in a vehicle. The light source configurator may be configured to take into account the relative locations of the first light source and the at least one camera when determining the light source configuration, to ensure that light emitted from the at least one light source is precisely directed onto the skin patch.

The at least one camera may be adjustable. In particular, the at least one camera may be adjustable to focus onto the at least one skin patch when capturing the second set of image frames. Similarly, in embodiments with first and second cameras, the second camera may be adjustable, for example to focus onto the at least one skin patch when capturing the second set of image frames.

The system may be installed in a vehicle. Accordingly, any reference to a ‘user’ in this disclosure is intended to include a driver or passenger in a vehicle. For example, the system may be connected to or integrated in an in-vehicle driving assistance system. Further, the system may be configured to provide an output signal indicative of the rPPG results, associated heartbeat characteristics, or an associated condition. The output signal can be received and processed by the in-vehicle driving assistance system, for example to generate a warning to the driver or to control the vehicle.

According to another aspect of the present disclosure, there is provided a computer program product including a computer-readable storage medium including instructions that, when executed by a processor, cause the processor to perform the method of the present disclosure, using the system of the present disclosure.

### BRIEF DESCRIPTION OF THE DRAWINGS

The features, objects, and advantages of the present disclosure will become more apparent from the detailed description set forth below when taken in conjunction with the drawings in which like reference numerals refer to similar elements.

FIG. 1 schematically illustrates a system for determining one or more heartbeat characteristics according to a first embodiment;

FIG. 2 schematically illustrates a system for determining one or more heartbeat characteristics according to a second embodiment;

FIG. 3 is another schematic illustration of the system according to the second embodiment in a different operation mode; and

FIG. 4 illustrates a flow chart of a method according to an embodiment.

### DETAILED DESCRIPTION OF EXEMPLARY EMBODIMENTS

The present disclosure provides a method that reduces the effect of environmental lighting when applying rPPG to determine heartbeat characteristics of a user of a vehicle (e.g., a driver or a passenger). The method generally includes identifying an area of the user's skin (also termed a ‘skin patch’ herein) and illuminating that area, before capturing a series of images for analysis using rPPG. The present disclosure also provides systems configured to execute the method disclosed herein.

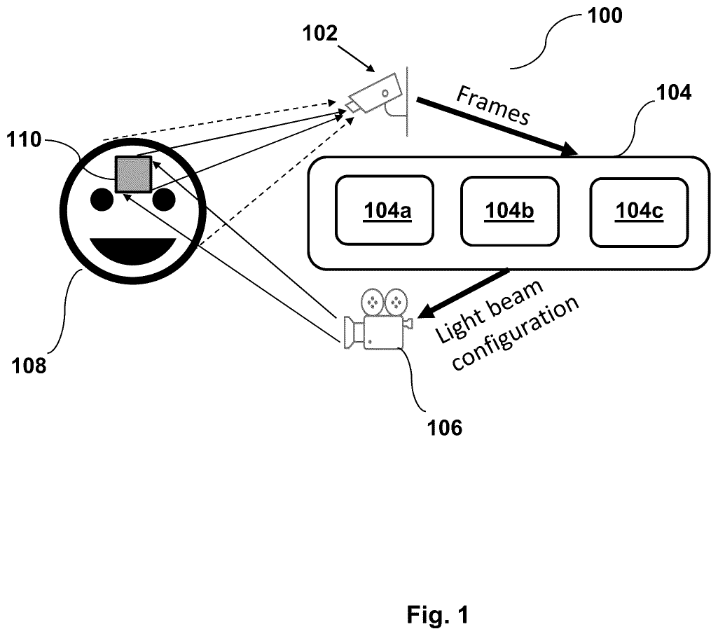

With reference to FIG. 1, a system 100 according to the first embodiment includes a camera 102, a computing system 104, and a light source 106. The computing system 104 includes a facial feature detector 104a, a light source configurator 104b and an rPPG system 104c. Operation of these components will be described in more detail below.

The camera 102 has an RGB sensor capable of capturing a series of image frames, using the visible light spectrum, with a resolution sufficient to allow facial contours and features, such as locations of the eyes, eyebrows, nose, and mouth, to be ascertained. When captured, each of the series of image frames is composed of a plurality of pixels and represents a face 108 of a user. Each pixel can have associated red-green-blue (RGB) values. Capturing visible light, and thereby allowing RGB values to be associated with the pixels, allows the color component which is best suited for rPPG for further analysis to be selected.

In an illustrative example, the series of image frames, which hereinafter may also be referred to as the sets of frames, are captured at a frame rate of 30 frames per second, fps, or 60 fps. The camera may have a horizontal field of view of 60-70°.

In the illustrated embodiment, the camera 102 is configured to transmit the series of image frames to the computing system 104. The computing system 104 is configured to receive the series of image frames and process them using the facial feature detector 104a. In particular, the facial feature detector 104a analyzes the series of image frames to identify an arrangement of pixels in each image frame that represents a skin patch 110 of the user's face 108. This can be done by detecting pixels corresponding to facial landmarks, in particular 3D facial landmarks, and then determining the arrangement of the pixels corresponding to the skin patch 110 relative to the detected facial landmarks. Examples of suitable facial landmarks include eyebrows, eyes, nose and mouth.

As an illustrative, non-limiting, example, the facial feature detector 104a can detect pixels corresponding to the eyes of the user 108, and the skin patch 110 may be defined as being an area of skin 1 cm by 1 cm located on the forehead with the lower left-hand corner being 3 cm from the left eye and the lower right-hand corner being 3 cm from the right eye.

The detection of pixels corresponding to facial landmarks can be implemented using known feature detection methods, which will not be described in further detail herein.

The facial feature detector 104a is configured to transmit information indicating the location of the skin patch 110 to the light source configurator 104b. Based on this information, the light source configurator 104b can calculate light source configurations and generate control signals to configure the light source 106 to illuminate the skin patch 110. The configuration of the light source 106 can relate to a direction, intensity and/or focusing of emitted light beams. Also, the configuration of the light source 106 can be selected to ensure that the user does not notice or is not distracted by the illumination, and that no light reaches the eye. Thus, the light source 106 is configured to illuminate the skin patch 110 using preferred illumination parameters.

The light source 106 may include, but is not limited to, a device with multiple emissive sources, for example several LEDs configured to have different light emission directions/angles and/or a device with electronic, electromechanical or mechanical control of the light beam, for example an LCD or DLP projector. The emission spectrum of the light source 106 may be selected to be suitable for rPPG processing. In an example, light emitted by the light source 106 includes green light. Green light reflected from the skin patch 110 has a good signal-to-noise ratio with respect to skin color changes caused by changes in the blood flow.

Also, the light source 106 may emit in a spectrum which matches the sensitivity profile of the camera 102 to ensure that differences (e.g., in color and/or intensity) of the emissions reflected from the skin patch 110 are more reliably captured by the camera 102. A calibration procedure can be employed to adjust the emitted light spectrum to correspond to the camera's sensitivity profile. Alternatively or additionally, the spectrum of the light source 106 may be adjusted to better match the skin color of the user.

In the illustrated embodiment, the rPPG system 104c is configured to receive image frames from the camera 102 and perform rPPG processing. In particular, rPPG system 104c may be configured to perform rPPG processing with respect to a subset of the pixels in the received series of image frames, the subset corresponding to the skin patch 110 identified by facial feature detector 104a, as described above. RPPG processing includes detecting changes in corresponding pixels of consecutive image frames, for example changes in color and/or intensity. Such changes may be caused by momentary changes in the blood flow underneath the skin patch 110. This may provide information about heartbeat parameters of the user 108 including, but not limited to heartbeat rate, heartbeat strength, heartbeat rhythm and inter-beat intervals.

The rPPG system 104c may also be capable of processing the rate of change of pixels in consecutive frames. This may be indicative of the rate of change or variability of the above heartbeat parameters.

The result of rPPG processing may be output by the rPPG system 104c. The output may be used to control another system, such as an in-vehicle driving assistance system. For example, the output by the rPPG system 104c may be used to generate a warning signal, to adapt an in-vehicle illumination, or to activate in-vehicle safety features. However, the present disclosure is not limited in this regard, and other applications and functions are envisaged.

Each of the facial feature detector 104a, the light source configurator 104b and the rPPG system 104c can be implemented by software instructions stored in a memory of the computing system 104, to be called for execution by a processor of the computing system 104. Alternatively, each of the facial feature detector 104a, the light source configurator 104b and the rPPG system 104c can include circuitry, such as an integrated circuit, of the computing system 104. Also, the facial feature detector 104a, the light source configurator 104b and the rPPG system 104c are shown as separate functional components in FIGS. 1-3. However, the present disclosure is not limited in this regard, and these components may be combined.

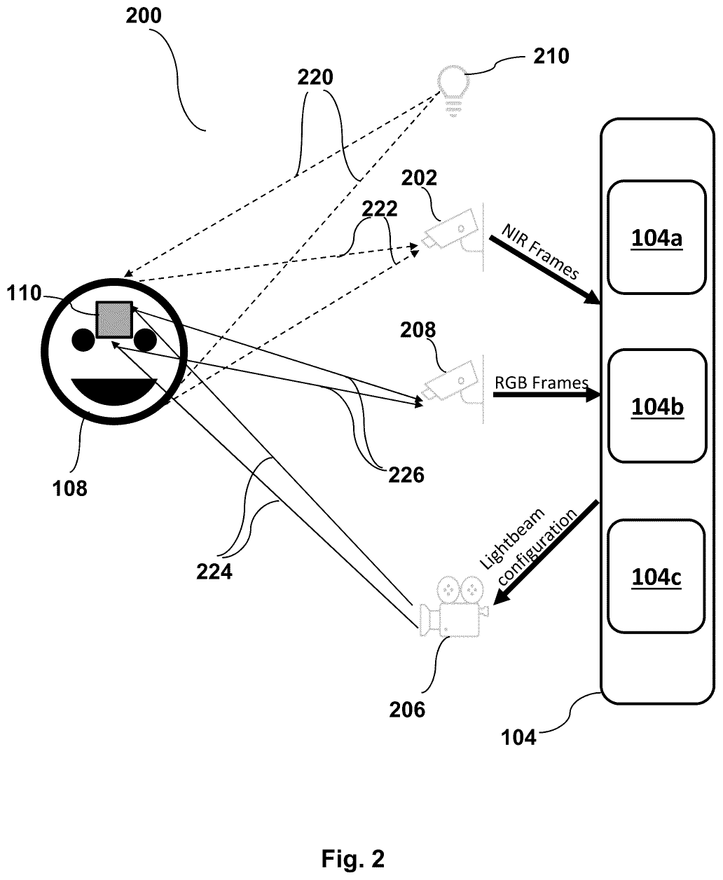

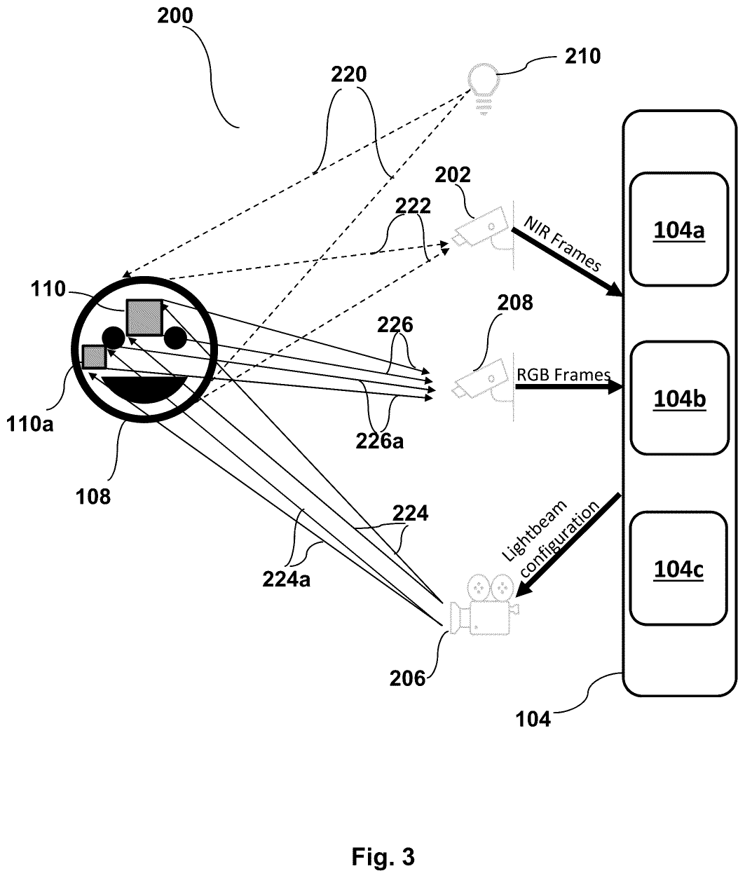

A system 200 according to the second embodiment of the present disclosure is shown in FIGS. 2 and 3. The system 200 has first and second cameras 202 and 208, rather than a single camera 102 (first embodiment). The first camera 202 may have an NIR sensor and is capable of capturing images in the NIR spectrum. The second camera 208 may have an RGB sensor and is capable of capturing images in the visible light spectrum. The second camera 208 may be identical to the camera 102 of the first embodiment.

In addition, the system 200 according to the second embodiment includes first and second light sources 206 and 210. The first light source 206 may be operable in the same way as the light source 106 of the first embodiment. The second light source 210 may be an infrared light source, in particular an NIR light source.

The second light source 210 may be operated to emit NIR light onto the face of the user 108, as indicated by dotted arrows 220. The infrared light may be reflected onto the NIR sensor of the first camera 202, as indicated by dotted arrows 222. The NIR sensor of the first camera 202 may be configured to generate a series of image frames based on the received NIR light. Similar to the camera 102 of the first embodiment, the first camera 202 is configured to transmit the series of image frames to the computing system 104. The computing system 104 can process the image frames using the facial feature detector 104a to determine the location of the skin patch 110. The operation of the facial feature detector 104a may be the same as in the first embodiment. Similarly, the operation of the light source configurator 104b may correspond to the first embodiment. In particular, the light source configurator 104b can calculate light source configurations and generate control signals to configure the light source 106 to illuminate the skin patch 110 with RGB light, as indicated by solid-line arrows 224. RGB light reflected from the skin 110 is captured by the second camera 208, as illustrated by solid-line arrows 226. The second camera 208 is configured to generate a series of RGB image frames based on the detected RGB light 226, and to transmit the series of RGB image frames to the rPPG system 104c. The operation of the rPPG system 104c of the second embodiment may also correspond to the first embodiment.

As described above, the second embodiment may differ from the first embodiment by including an additional infrared light source (the second light source 210) and an additional camera having an NIR sensor (the first camera 202). These additional components enable performing the detection of facial features, as described above, using infrared light. This may enable the facial features of the user's face 108 to be more accurately identified in poor lighting conditions, thereby improving identification of the skin patch 110. Also, since infrared light is invisible to the user, no distraction or inconvenience is caused.

The systems 100 and 200 of the first and second embodiments may also be operated to identify and illuminate two or more skin patches. FIG. 3 illustrates an example of the system 200 according to the second embodiment, wherein the second camera 206 is operated to illuminate two skin patches 110 and 110a. The skin patches 110 and 110a may be located in different regions of the face 108 and have different sizes. In an example, only one of the skin patches 110, 110a is selected for further processing. The selection may be performed based on one or more criteria including signal-to-noise ratios, size and homogeneity of the skin patches and head position.

The second light source 206 may include multiple light emissive sources, for example several LEDs, to enable illuminating the two skin patches 110 and 110a at the same time. Alternatively, the two skin patches 110 and 110a may be illuminated consecutively or alternatingly. Illuminating two more skin patches can increase the accuracy of the rPPG processing by the rPPG system 104c.

In an alternative embodiment, the first and second cameras 202 and 206 may be combined to form a single, integrated camera having an NIR sensor and an RGB sensor. This is beneficial when the system is deployed in the cabin of a vehicle where space can be limited. Further, a single camera having the capability to capture both RGB and NIR images will not experience any issues based on the relative location of a NIR sensor to a RGB sensor used to identify facial features and to enable rPPG, respectively.

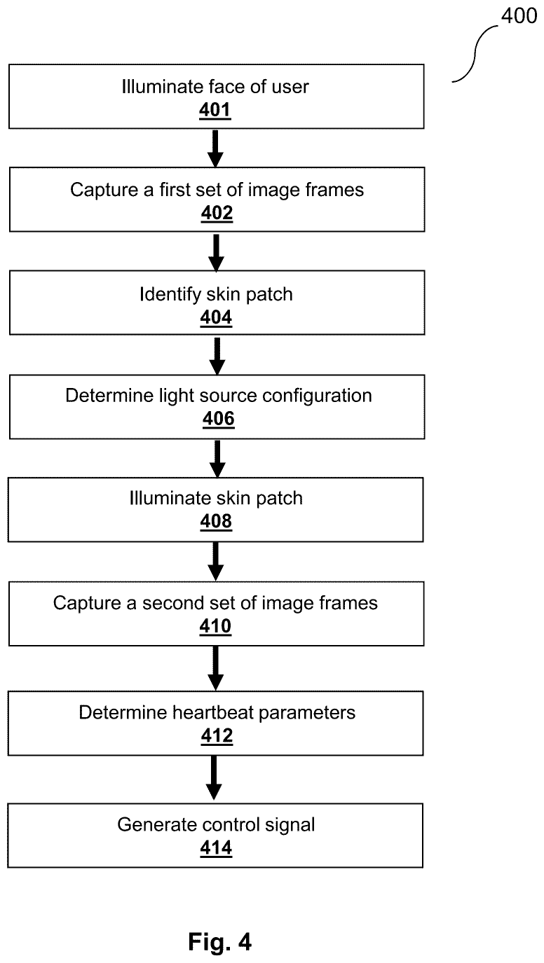

A method 400 according to an embodiment of the present disclosure is shown in FIG. 4. Whilst the method 400 can be performed using any suitable system, the following example will be described with references to the systems 100 and 200 of FIGS. 1-3.

At step 401, the face 110 of the user is illuminated. The illumination can be provided by natural background light and/or an in-vehicle illumination system. Alternatively, or in addition, the illumination can be provided by illuminating the face 110 using a light source such as the second light source 210 of system 200. The face 110 may be illuminated using visible light and/or infrared light.

At step 402, a first set of image frames that include a representation of the user's face 108 is captured. The first set of image frames may be captured using the visible light spectrum, for example using the camera 102 of the system shown in FIG. 1, or using the NIR spectrum, for example using the first camera 202 of the system shown in FIGS. 2 and 3. When using the NIR spectrum, the user's face 108 is illuminated with infrared light from the light source 210.

At step 404, the frames of the first set of image frames are analyzed to identify a representation of the skin patch 110 in each of the frames. Identifying the representation of the skin patch 110 in the frames includes recognizing a region (such as a group of adjacent pixels) in each frame that represents the skin patch 110. Recognizing a region in a frame that represents the skin patch 110 may include identifying one or more reference pixels, which correspond to reference points on the user's face 108 (such as the eyebrows, the eyes, the nose or the mouth), and calculating a relative position of the one or more reference pixels to the region. Optionally, more than one region can be recognized in each frame, with each region representing an associated skin patch (e.g., skin patches 110 and 110a). If more than one region is recognized, a subset of the recognized regions can be selected for further processing based on various factors including the number of pixels in the region and/or signal-to-noise ratios.

At step 406, a light source configuration is determined. The light source configuration is transmitted to a light source (e.g., the light source 106 in system 100 or the first light source 206 in system 200) in a light source configuration signal. The light source configuration can include an emission direction, an emission intensity and an emission wavelength for the light beams emitted by the light source 106 (first light source 206).

As part of step 406, determining the light source configuration may take into account the relative locations of the user's face 108, the camera 102 (first camera 202) and the light source 106 (first light source 206) to one another. In particular, step 406 may take into account a relative location of the camera 102 (first camera 202) to the user's face, as well as a relative location of the light source 106 (first light source 206) to the user's face 108. Based on the relative locations, a target direction for a light beam from the light source 106 (first light source 206) may be determined.

At step 408, the light source 106 (first light source 206) is operated to illuminate the skin patch 110 using the light source configuration determined in step 406.

At step 410, a second set of image frames is captured. Step 410 may be performed by the camera 102 of system 100 or the second camera of the system 200.

At step 412, the frames of the second set of image frames are analysed using rPPG, to determine one or more heartbeat parameters. As described above, this may include determining changes between pixels in consecutive frames of the second set of image frames, for example changes in colour or intensity. The changes may be indicative of heartbeat parameters such as heartbeat rate. This, in turn may be indicative of physiological or psychological conditions that may affect driving ability.

If the method is performed using a system having two cameras, such as system 200, step 412 may include taking into account the relative locations of the first and second cameras 202 and 208, in order to more accurately determine the location of the illuminated skin patch 410 (and 410a) in the captured second set of image frames. As part thereof, coordinates of the skin patch 410 (and 410a) in the first set of image frames may be translated into coordinates in the second set of image frames.

At step 414, the method may include generating a control signal to initiate an action associated with a heartbeat parameter determined in step 412. Examples of such action include, but are not limited to, generating a visual and/or audible indication to the user, changing an in-vehicle illumination, activating an assisted driving function such as lane keeping, adapting an in-vehicle air conditioning system, etc.

Some or all of the above steps may be repeated or performed continuously, to enable an ongoing calibration and/or rPPG analysis and to allow appropriate responsive action, as described above.

The systems and method of the present disclosure enable performing a more accurate rPPG analysis, particularly in environments with fluctuating light conditions such as a vehicle. The systems and method of the present disclosure achieve this by identifying one or more skin patches on a user's face that are suitable for rPPG analysis, and illuminating the identified skin patches while performing rPPG. Thereby, the effects of fluctuating light can be reduced or eliminated.

The description of embodiments and aspects has been presented merely for purposes of illustration and description. Suitable modifications and variations to there embodiments and aspects may be performed in light of the above, and different embodiments and aspects may be combined where possible and appropriate, without departing from the scope of protection as determined by the claims.

The invention claimed is:

1. A method comprising: capturing a first set of image frames, wherein the first set of image frames includes a representation of a user's face; identifying at least one skin patch of the user's face that is represented in the first set of frames; determining a light source configuration and transmitting the light source configuration to a first light source; illuminating, by the first light source, the at least one skin patch according to the light source configuration; capturing a second set of image frames, wherein the second set of image frames includes a representation of the at least one skin patch illuminated by the first light source according to the light source configuration; and processing one or more of the second set of image frames using remote photo-plethysmography, rPPG.

2. The method of claim 1, wherein the capturing of the first and second sets of image frames is performed by a single camera.

3. The method of claim 2, wherein the camera comprises a red-green-blue, RGB, sensor, and wherein the capturing of the first and second sets of image frames comprises capturing images in the visible light spectrum using the RGB sensor.

4. The method of claim 2, wherein the camera comprises a near-infrared, NIR, sensor, and an RGB sensor, wherein the capturing of the first set of image fames comprises capturing images in the NIR spectrum using the NIR sensor, and wherein the capturing of the second set of image frames comprises capturing images in the visible light spectrum using the RGB sensor.

5. The method of claim 1, wherein the capturing of the first set of image frames is performed by a first camera, and wherein the capturing of the second set of image frames is performed by a second camera.

6. The method of claim 5, wherein the first camera comprises an NIR sensor and the first set of image frames is captured in the NIR spectrum, and wherein the second camera comprises an RGB sensor and the second set of image frames is captured in the visible light spectrum.

7. The method of claim 4, further comprising:
illuminating the user's face with a NIR light source to facilitate identifying the at least one skin patch.

8. The method of claim 1, further comprising:
selecting at least some of the second set of image frames for rPPG processing, wherein the selecting is based on one or more quality criteria, for example a signal-tonoise ratio.

9. The method of claim 1, wherein identifying the at least one skin patch comprises:
detecting one or more facial features represented in the first set of image frames; and
identifying the at least one skin patch based on a location of the detected one ore more facial features.

10. The method of claim 1, further comprising:
determining one or more heartbeat parameters based on the rPPG processing; and
generating a control signal based on the one or more heartbeat parameters to initiate an action associated with the one or more heartbeat parameters.

11. A system comprising:
at least one camera for capturing a first set of image frames, wherein the first set of image frames includes a representation of a user's face;
a facial feature detector for identifying at least one skin patch of the user's face that is represented in the first set of image frames; a light source configurator for determining a light source configuration; and
a first light source for illuminating the at least one skin patch according to the light source configuration;
wherein the at least one camera is configured to capture a second set of image frames, wherein the second set of image frames includes a representation of the at least one skin patch illuminated by the first light source according to the light source configuration, and wherein the system further comprises a remote photoplethysmography, rPPG, system for processing one or more of the second set of image frames using rPPG.

12. The system of claim 11, wherein the at least one camera comprises a red-green-blue, RGB, sensor for capturing the first and second sets of image frames.

13. The system of claim 11, wherein the at least one camera comprises a near-infrared, NIR, sensor for capturing the first set of image frames in the NIR spectrum, and an RGB sensor for capturing the second set of image frames in the visible light spectrum.

14. The system of claim 11, wherein the at least one camera comprises a first camera for capturing the first set of image frames, and a second camera for capturing the second set of image frames.

15. The system of claim 14, wherein the first camera comprises an NIR sensor for capturing the first set of image frames in the NIR spectrum, and wherein the second camera comprises an RGB sensor for capturing the second set of image frames in the visible light spectrum.

16. The system of claim 13, further comprising:
an NIR light source for illuminating the user's face with NIR light.

17. The system of claim 11, wherein the first light source is controllable to illuminate more than one skin patch on the user's face separately and/or independently of one another.

18. The system of claim 11, wherein the first light source comprises a plurality of light emission sources, for example a plurality of light emitting diodes, LEDs, wherein some or all of the plurality of light emission sources are individually configurable.

19. The system of claim 11, wherein the wherein the first light source comprises a liquid crystal display, LCD, projector or a digital light processing, DLP, projector.

20. (canceled)
